(19) United States
(12) Patent Application Publication
(10) Pub. No.: US 2006/0194854 A1
(43) Pub. Date: Aug. 31, 2006

(54) New 2-substituted - 1,3-thiazole compounds

(75) Inventors: Stefan Berg, Sodertalje (SE); Sven Hellberg, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/430,061

(22) Filed: May 8, 2006

Related U.S. Application Data
(63) Continuation of application No. 10/510,846, filed on Oct. 12, 2004, now abandoned.

(30) Foreign Application Priority Data
Apr. 19, 2002 (SE) 0201194-8

(51) Int. Cl. C07D 277/04; A61K 31/426

(52) U.S. Cl. 514/370; 548/191

(57) ABSTRACT

The present invention relates to new compounds of formula I, Wherein Y is NR⁴CONR⁴, NR⁴CO, or NR⁴; R¹ is nitro or COR⁵; R² is hydrogen or NH₂; R³ is C₁₋₆alkyl or C₀₋₆akylaryl wherein C₀₋₆alkylaryl may be substituted by A; R⁴ is hydrogen; R⁵ is C₁₋₆alkyl; A is independently selected from halo, OR⁶ and C₁₋₆alkyl; R⁶ is C₁₋₆alkyl; provided that the compound is not N-(4-Methoxybenzyl)-N′-(5-nitro-1,3-thiazol-2-yl)urea as a free base or a salt thereof as well as a process for their preparation, pharmaceutical formulations containing said therapeutically active compounds and to the use of said active compounds in therapy.

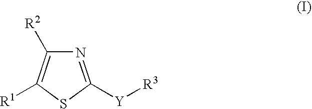

### FIELD OF THE INVENTION

The present invention relates to new compounds of formula I as a free base or a pharmaceutically acceptable salt thereof, to pharmaceutical formulations containing said compounds and to the use of said compounds in therapy. The present invention further relates to a process for the preparation of compounds of formula I.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but believed to be related. Glycogen synthase kinase 3β (GSK3β) or Tau (τ) phosphorylating kinase selectively phosphorylates the microtubule associated protein c in neurons at sites that are hyperphosphorylated in AD brains. Hyperphosphorylated protein r has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of c and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

Chronic and Acute Neurodegenerative Diseases.

Growth factor mediated activation of the PI3K/Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

Schizophrenia

GSK3 is involved in signal transduction cascades of multiple cellular processes, particularly during neural development. Kozlovsky et al (Am J Psychiatry 2000 May;157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February;49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25;95 (5):605-14)). The new follicles formed sebaceous glands and dermal papilla, normally established only in embryogenesis. Thus GSK3 inhibition may offer treatment for baldness.

Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### DISCLOSURE OF THE INVENTION

The object of the present invention is to provide compounds having a selective inhibiting effect of GSK3 as well as having a good bioavailability.

Accordingly, the present invention provides a compound of formula I 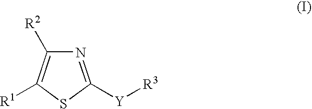
wherein:
Y is NR⁴CONR⁴, NR⁴CO, or NR⁴;
R¹ is nitro or COR⁵;
R² is hydrogen or NH₂;
R³ is C₁₋₆alkyl or C₀₋₆alkylaryl wherein C₀₋₆alkylaryl may be substituted by A;
R⁴ is hydrogen;
R⁵ is C₁₋₆alkyl;
A is independently selected from halo, OR⁶ and C₁₋₆alkyl;
R⁶ is C₁₋₆alkyl;
provided that the compound is not N-(4-Methoxybenzyl)-N′-(5-nitro-1,3-thiazol-2-yl)urea; as a free base or a salt thereof.

The compound N-(4-Methoxybenzyl)-N′-(5-nitro-1,3-thiazol-2-yl)urea is known and is disclosed in WO 03/004478.

One embodiment of the invention relates to compounds of formula I wherein Y is NR⁴CONR⁴ or NR⁴CO.

Another embodiment of the invention relates to compounds of formula I wherein Y is NR⁴.

Yet another embodiment of the invention relates to compounds of formula I wherein R¹ is nitro.

Yet another embodiment of the invention relates to compounds of formula I wherein R¹ is COR⁵.

Yet another embodiment of the invention relates to compounds of formula I wherein R⁵ and R⁶ is methyl.

Yet another embodiment of the invention relates to compounds of formula I wherein R² is hydrogen.

Yet another embodiment of the invention relates to compounds of formula I wherein R² is is NH₂.

Yet another embodiment of the invention relates to compounds of formula I wherein R³ is C₁₋₃alkyl or phenyl, said phenyl optionally being substituted with A.

Yet another embodiment of the invention relates to compounds of formula I wherein R³ is phenyl, substituted with A; A being OR⁶ and R⁶ being methyl.

One aspect of the invention relates to the following compounds;
N-Butyl-N′-(5-nitro 1,3-thiazol-2-yl)urea;
N-(5-Nitro-1,3-thiazol-2-yl)pentanamide;
1-{4-Amino-2-[(4-methoxyphenyl)amino]-1,3-thiazol-5-yl}ethanone; N-Benzyl-N′-(5-nitro-1,3-thiazol-2-yl)urea;
3-(4-Methoxyphenyl)-N-(5-nitro-1,3-thiazol-2-yl)propanamide;
4-(4-Methoxyphenyl)-N-(5-nitro-1,3-thiazol-2-yl)butanamide;
2-(3-Methoxyphenyl)-N-(5-nitro-1,3-thiazol-2yl)acetamide;
2-(4-Fluorophenyl)-N-(5-nitro-1,3-thiazol-2-yl)propanamide;
2-(3-Methylphenyl)-N-(5-nitro-1,3-thiazol-2-yl)acetamide;
   as a free base or a salt thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

For the avoidance of doubt it is to be understood that in this specification ‘C₀₋₆’ means a carbon group having 0, 1, 2, 3, 4, 5 or 6 carbon atoms.

In this specification, unless stated otherwise, the term “alkyl” includes both straight and branched chain alkyl groups and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl.

In this specification, unless stated otherwise, the term “C₀₋₆ alkylaryl”, includes both substituted and unsubstituted alkylaryl groups, which may be substituted on the alkyl and/or the aryl and may be, but are not limited to, C₁₋₃alkylphenyl, such as benzyl, ethylphenyl, or propylphenyl

In the case where a subscript is the integer 0 (zero) the group to which the subscript refers to, indicates that the group is absent, i.e. there is a direct bond between the groups.

In this specification, unless stated otherwise, the term “Halo” refers to halogen and may be fluorine, chlorine, bromine or iodine.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

Both organic and inorganic acids can be employed to form non-toxic pharmaceutically acceptable salts of the compounds of this invention. Pharmaceutically acceptable salts include, but are not limited to hydrochloride. These salts are readily prepared by methods known in the art.

Some compounds of formula I may have chiral centres and/or geometric isomeric centres (E- and Z-isomers), and it is to be understood that the invention encompasses all such optical, diastereoisomeric and geometric isomers.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

Pharmaceutical Compositions

According to one aspect of the present invention there is provided a pharmaceutical composition comprising a compound of formula I, as a free base or a pharmaceutically acceptable salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt thereof, can be used on its own but will usually be administered in the form of a pharmaceutical composition in which the formula I compound/salt (active ingredient) is in association with a pharmaceutically acceptable diluent or carrier. Dependent on the mode of administration, the pharmaceutical composition may comprise from 0.05 to 99% w (percent by weight), for example from 0.10 to 50% w, of active ingredient, all percentages by weight being based on total composition.

A diluent or carrier includes water, aqueous polyethylene glycol, magnesium carbonate, magnesium stearate, talc, a sugar (such as lactose), pectin, dextrin, starch, tragacanth, microcrystalline cellulose, methylcellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt thereof, a hereinbefore defined, with a pharmaceutically acceptable diluent or carrier.

An example of a pharmaceutical composition of the invention is an injectable solution containing a compound of the invention, or a a pharmaceutically acceptable salt thereof, as hereinbefore defined, and sterile water, and, if necessary, either sodium hydroxide or hydrochloric acid to bring the pH of the final composition to about pH 5, and optionally a surfactant to aid dissolution.

Liquid solution comprising a compound of formula I, or a salt thereof, dissolved in water.

| Solution | mg/mL |
|---|---|
| Active Compound | 5.0% w/v |
| Pure water | To 100% |

Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting predemented states, Mild Cognitive Impairment, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies and androgenetic alopecia.

One embodiment of the invention relates to the prevention and/or treatment of dementia and Alzheimer's Disease.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administrering to a mammal, including man in need of such treatment and/or prevention a therapeutically effective amount of a compound of formula I, as hereinbefore defined.

Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

Methods of Preparation

The processes for the preparation of a compound of formula I, wherein halo, R¹, R², R³ and R⁵ unless otherwise specified, are as defined hereinbefore, comprising:
(i) reacting a compound of formula II, wherein R² is hydrogen, with a compound of formula III, 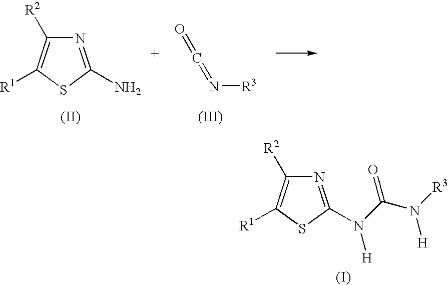
(ii) reacting a compound of formula II, wherein R² is hydrogen, with an activated carboxylic acid R³COL, wherein L is a leaving group such as Halo; 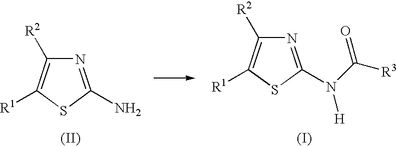
(iii) by using a carboxylic acid, R³COOH with an activating reagent such as N,N′-carbonyldiimidazole or N,N′-dicyclohexylcarbodiimide in a suitable solvent such as N,N-dimethylformamide or tetrahydrofuran and the reaction may be conducted at a temperature between +20° C. and +150° C.;
(iv) reacting a compound of formula IV, wherein R³ is C₁₋₆alkyl or C₀₋₆alkylaryl, with a compound of formula V, wherein R⁵ is C₁₋₆alkyl and Halo is chloro or bromo.

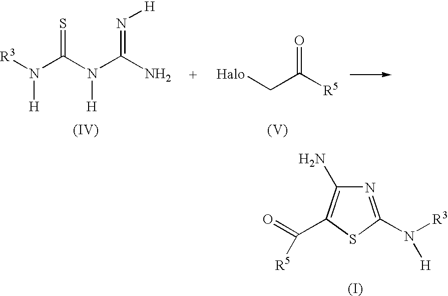

### EXAMPLES

The invention will now be illustrated in the following non-limiting Examples and unless stated otherwise:

(i) temperatures are given in degrees Celsius (° C.); operations were carried out at room or ambient temperature, i.e. at a temperature in the range of 18 to 25° C.;

(ii) yields are given for illustration only and are not necessarily those which can be obtained by diligent process development; preparations were repeated if more material was required;

(iii) chemical symbols have their usual meanings; SI units and symbols are used;

(iv) solvent ratios are given in volume:volume (v/v) terms; and

(v) all starting materials are commercially available or earlier described in the literature.

### Example 1

### N-Butyl-N′-(5-nitro-1,3-thiazol-2-yl)urea

To a solution of 2-amino-5-nitrothiazole (145 mg, 1 mmol) in N,N′-dimethylformamide (15 mL) was added butyl isocyanate (99 mg, 1 mmol) and a catalytic amount of potassium tert-butoxide. The reaction mixture was stirred at 100° C. for 6 h. The solvent was removed in vacuo and the residue was taken up in ethyl acetate and washed with water. The organic layer was dried with magnesium sulfate, filtered and concentrated. The crude product was purified on a silica gel column using hexane/ethyl acetate (3:1) as the eluent to give 120 mg (49% yield) of the title compound as a solid: ¹H NMR (DMSO-d₆, 400 MHz) δ 8.49 (s, 1H), 6.81 (br s, 1H), 3.34 (br s, 1H), 3.15 (q, J=7 Hz, 2H), 1.47-1.40 (m, 2H), 1.34-1.24 (m, 2H), 0.88 (t, J=7 Hz, 3H); ¹³C NMR (DMSO-d₆, 100 MHz) δ 164.37, 153.26, 143.48, 140.78, 39.17, 31.31, 19.41, 13.62; MS (ESP) m/z 243.0 (M⁺+1).

### Example 2

### N-(5-Nitro-1,3-thiazol-2-yl)pentanamide

To a solution of 2-amino-5-nitrothiazole (205 mg, 1.41 mmol) and triethylamine (271 μL, 2.11 mmol) in methylene chloride (25 mL) was added dropwise n-valeroylchloride (180 μL, 1.48 mmol). The reaction solution was stirred over night and washed with a saturated sodium bicarbonate solution. The layers were separated and the organic layer was dried with sodium sulfate, filtered and concentrated. The crude product was purified on a silica gel column using hexane/ethyl acetate (4:1) as the eluent to give 130 mg (40% yield) of the title compound as a light yellow solid: mp 155-156° C.; ¹H NMR (CDCl₃, 300 MHz) δ 11.2 (br s, 1H), 8.29 (s, 1H), 2.59 (t, J=7 Hz, 2H), 1.84-1.74 (m, 2H), 1.51-1.39 (m, 2H), 0.98 (t, J=7 Hz, 3H); ¹³C NMR (CDCl₃, 75 MHz) δ 171.67, 162.02, 143.46, 139.46, 35.98, 26.38, 22.24, 13.67; EIMS (70 eV) m/z (relative intensity) 229 (M⁺, 34), 85 (100), 57 (24).

### Example 3

### 1-{4-Amino-2-[(4-methoxyphenyl)amino]-1,3-thiazol-5-yl}ethanone

To a solution of 1-(4-methoxyphenyl)-3-amidino-2-thiourea (204 mg, 0.91 mmol) in acetone (5 mL) was added chloroacetone (84 mg, 0.91 mmol) in acetone (2 mL). The resulting solution was heated at 50° C. and triethylamine (110 μL, 1.09 mmol) was added. After 5 min, ethanol (5 mL) was added to prevent precipitation in the reaction solution. After an additional 35 min at 50° C., the solvents were removed in vacuo. The resulting yellow oil was partitioned between ethyl acetate and water. The layers were separated and the organic layer was washed with brine, dried with magnesium sulfate, filtered and concentrated to give 134 mg (56% yield) of the title compound as a beige solid: mp 180° C. (decomp.); ¹H NMR (DMSO-d₆, 400 MHz) δ 10.50 (br s, 1H), 7.69 (br s, 2H), 7.48 (d, J=9 Hz, 2H), 6.92 (d, J=9 Hz, 2H), 3.73 (s, 3H), 2.05 (s, 3H); ¹³C NMR (DMSO-d₆, 100 MHz) δ 184.88, 166.27, 163.47, 155.66, 132.82, 121.82, 114.32, 55.27, 28.93; MS (APcI) m/z 264 (M⁺+1).

### Example 4

### N-Benzyl-N′-(5-nitro-1,3-thiazol-2-yl)urea

A solution of 2-amino-5-nitrothiazole (273 mg, 1.88 mmol) and benzyl isocyanate (275 mg, 2.07 mmol) in anhydrous N,N-dimethylformamide (5 mL) was stirred at 100° C. under nitrogen atmosphere for 17 h. The solvent was removed in vacuo and the residue was partitioned between ethyl acetate and water. The layers were separated and the organic layer was washed with brine, dried with magnesium sulfate, filtered and concentrated to give a yellow oil. The crude product was purified on a silica gel column using chloroform/ethanol, (97:3), as the eluent to give 275 mg of the title compound as a yellow solid. The solid was purified from ethyl acetate/isopropanol to give 55 mg (53% yield) of the title compound: mp 210-211° C. (decomp.); ¹H NMR (DMSO-d₆, 400 MHz) δ 11.57, (br s, 1H), 8.35 (s, 1H), 7.20-7.08 (m, 6H), 4.21 (d, J=6 Hz, 2H); ¹³C NMR (DMSO-d6, 100 MHz) δ 164.37, 153.51, 143.45, 140.87, 138.95, 128.44, 127.22, 127.08, 43.11; is MS (TSP) m/z 279 (M⁺+1).

### Example 5

### 3-(4-Methoxyphenyl)-N-(5-nitro-1,3-thiazol-2-yl)propanamide

The compound was prepared as described for Example 5 using 3-(4-methoxyphenyl)propanoyl chloride. The reaction mixture was washed with a saturated aqueous sodium bicarbonate solution, dried with magnesium sulfate, filtered and concentrated. The crude product was purified on a silica gel column using heptane/ethyl acetate (3:2) as the eluent to give the title compound. Yield 10%: mp 224-227° C. (decomp.); ¹H NMR (DMSO-d₆, 400 MHz) δ 13.09 (br s, 1H), 8.62 (s, 1H), 7.14 (d, J=9 Hz, 2H), 6.84 (d, J=9 Hz, 2H), 3.70 (s, 3H), 2.89-2.79 (m, 4H); ¹³C NMR (DMSO-d₆, 100 MHz) δ 172.42, 161.62, 157.69, 142.72, 141.71, 132.18, 129.23, 128.26, 113.81, 54.98, 36.96, 29.16; MS (TSP) m/z 308 (M⁺+1).

### Example 6

### 4-(4-Methoxyphenyl)-N-(5-nitro-1,3-thiazol-2-yl)butanamide

To a suspension of 2-amino-5-nitrothiazole (283 mg, 1.95 mmol) and triethylamine (180 μL, 1.30 mmol) in methylene chloride (100 mL) was added a solution of 4-(4-methoxyphenyl)butanoyl chloride (277 mg, 1.30 mmol) in methylene chloride (3 mL). The reaction mixture was stirred for 4 days at ambient temperature and washed with water. The organic layer was dried with magnesium sulfate, filtered and concentrated. The yellowish oil was recrystallized from ethyl acetate to give 161 mg (39% yield) of the title compound as a beige solid: mp 176-177° C.; ¹H NMR (DMSO-d₆, 400 MHz) δ 13.02 (br s, 1H), 8.60 (s, 1H), 7.12 (d, J=8 Hz, 2H), 6.84 (d, J=8 Hz, 2H), 3.71 (s, 3H), 2.57-2.51 (m, 4H), 1.93-1.88 (m, 3H); ¹³C NMR (DMSO-d₆, 100 MHz) δ 172.91, 161.70, 157.50, 142.65, 141.61, 133.03, 129.27, 113.70, 54.92, 34.03, 33.44, 26.05; EIMS m/z 321 (M⁺).

### Example 7

### 2-(3-Methoxyphenyl)-N-(5-nitro-1,3-thiazol-2yl)acetamide

A solution of 3-methoxyphenylacetic acid (196 mg, 1.18 mmol) and 1,1′-carbonyldiimidazole (191 mg, 1.18 mmol) in N,N-dimethylformamide (5 mL) was heated at 100° C. for 20 min. 2-Amino-5-nitrothiazole (171 mg, 1.18 mmol) was added, and the reaction mixture was heated at 100° C. for 2.5 h. The mixture was allowed to cool, and was then partitioned between ethyl acetate and water. The layers were separated and the organic layer was washed with brine, dried with magnesium sulfate, filtered and concentrated. Purification on a silica gel column using heptane/ethyl acetate, (65:35), as the eluent gave 103 mg (30% yield) of the title compound as a yellow solid: ¹H NMR (DMSO-d₆, 400 MHz) δ 13.31 (br s, 1H), 8.63 (s, 1H), 7.26 (t, J=8 Hz, 1H), 6.92-6.84 (m, 3H), 3.85 (s, 2H), 3.75 (s, 3H); ¹³C NMR (DMSO-d₆, 100 MHz) δ 171.00, 161.77, 159.36, 142.72, 141.95, 135.41, 129.59, 121.62, 115.26, 112.52, 55.08, 41.59; EIMS m/z 294 (M⁺).

### Example 8

### 2-(4-Fluorophenyl)-N-(5-nitro-1,3-thiazol-2-yl)propanamide

The compound was prepared as described for Example 8 using 3-(4-fluorophenyl)propionic acid. Yield: 24%: ¹H NMR (DMSO-d₆, 400 MHz) δ 13.08 (br s, 1H), 8.60 (s, 1H), 7.26 (dd, J=8, 6 Hz, 2H), 7.10 (t, J=9 Hz, 2H), 2.93 (t, J=7 Hz, 2H), 2.83 (t, J=7 Hz, 2H); ¹³C NMR (DMSO-d₆, 100 MHz) δ 172.26, 162.00, 161.61, 159.60, 142.68, 141.71, 136.51, 136.48, 130.11, 130.03, 115.18, 114.97, 36.68, 29.11; MS (TSP) m/z 296 (M⁺+1).

### Example 9

### 2-(3-Methylphenyl)-N-(5-nitro-1,3-thiazol-2-yl)acetamide

The compound was prepared as described for Example 8 using m-tolylacetic acid. Yield: 32%: ¹H NMR (DMSO-d₆, 400 MHz) δ 13.29 (br s, 1H), 8.62 (s, 1H), 7.22 (t, J=8 Hz, 1 H), 7.13-7.07 (m, 3H), 3.82 (s, 2H), 2.28 (s, 3H); ¹³C NMR (DMSO-d₆, 100 MHz) δ 171.03, 161.65, 142.58, 141.78, 137.54, 133.77, 129.88, 128.32, 127.61, 126.35, 41.37, 20.86; MS (TSP) m/z 279 (M⁺+1).

Pharmacology

Determination of ATP Competition in Scintillation Proximity GSK3β Assay.

GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK30 (Dundee University, UK), 12 mM morpholinepropanesulfonic acid (MOPS), pH 7.0, 0.3 nM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:
MOPS Morpholinepropanesulfonic acid
EDTA Ethylenediaminetetraacetic acid
BSA Bovin Serum Albumin
ATP Adenosine Triphosphate
SPA Scintillation Proximity Assay
GSK3 Glycogen Synthase Kinase 3
   Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.010 nM to about 300 nM.

1. A compound having the formula I 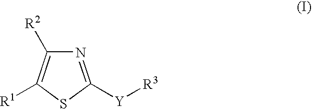
wherein:
Y is NR⁴CONR⁴, NR⁴CO, or NR⁴;
R¹ is nitro or COR⁵;
R² is hydrogen or NH₂;
R³ is C₁₋₆alkyl or C₀₋₆alkylaryl wherein C₀₋₆alkylaryl may be substituted by A;
R⁴ is hydrogen;
R⁵ is C₁₋₆alkyl;
A is independently selected from halo, OR⁶ and C₁₋₆alkyl;
R⁶ is C₁₋₆alkyl;
provided that the compound is not N-(4-Methoxybenzyl)-N′-(5-nitro-1,3-thiazol-2-yl)urea; as a free base or a salt thereof.

2. The compound according to claim 1, wherein Y is NR⁴CONR⁴ or NR⁴CO.

3. The compound according to claim 1, wherein Y is NR⁴.

4. The compound according to claim 1 or 2, wherein R¹ is nitro.

5. The compound according to claim 1 or 3, wherein R¹ is COR⁵.

6. The compound according to any one of claims 1 to 3, wherein R⁵ and R⁶ is methyl.

7. The compound according to any one of claims 1 and 2, wherein R² is hydrogen.

8. The compound according to any one of claims 1 and 3, wherein R² is is NH₂.

9. The compound according to any one of claims 1 to 3, wherein R³ is C₁₋₃alkyl or phenyl, said phenyl optionally being substituted with A.

10. The compound according to claim 9, wherein R³ is phenyl, substituted with A; A being OR⁶ and R⁶ being methyl.

11. A compound which is
N-Butyl-N′-(5-nitro-1,3-thiazol-2-yl)urea;
N-(5-Nitro-1,3-thiazol-2-yl)pentanamide;
1-{4-Amino-2-[(4-methoxyphenyl)amino]-1,3-thiazol-5-yl}ethanone;
N-Benzyl-N′-(5-nitro-1,3-thiazol-2-yl)urea;
3-(4-Methoxyphenyl)-N-(5-nitro-1,3-thiazol-2-yl)propanamide;
4-(4-Methoxyphenyl)-N-(5-nitro-1,3-thiazol-2-yl)butanamide;
2-(3-Methoxyphenyl)-N-(5-nitro-1,3-thiazol-2yl)acetamide;
2-(4-Fluorophenyl)-N-(5-nitro-1,3-thiazol-2-yl)propanamide;
2-(3-Methylphenyl)-N-(5-nitro-1,3-thiazol-2-yl)acetamide;
as a free base or a salt thereof.

12. A pharmaceutical formulation comprising as active ingredient a therapeutically effective amount of the compound of any one of claims 1, 2, 3 and 11 and pharmaceutically acceptable carriers or diluents.

13-22. (canceled)

23. A method of prevention and/or treatment of conditions associated with glycogen synthase kinase-3, comprising administering to a mammal, including man in need of such prevention and/or treatment, a therapeutically effective amount of a compound of formula I as defined in any one of claims 1, 2, 3 and 11.

24. The method according to claim 23, wherein the condition is one or more of dementia, Alzheimer's Disease, Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down's syndrome, Huntington's Disease, postencephelatic parkinsonism, progressive supranuclear palsy, Niemann-Pick's Disease, Pick's Disease, stroke, head trauma and other chronic neurodegenative diseases, Bipolar Disease, affective disorders, depression, schizophrenia, cognitive disorders, Type I and Type II diabetes, diabetic neuropathy, hair loss and contraceptive medication.

25. The method according to claim 24, wherein the condition is dementia and Alzheimer's Disease.

26. A process for the preparation of a compound of formula I according to claim 1, wherein halo, R¹, R², R³ and R⁵ unless otherwise specified, are defined as in claim 1, comprising:
(i) reacting a compound of formula II, wherein R² is hydrogen, with a compound of formula III, 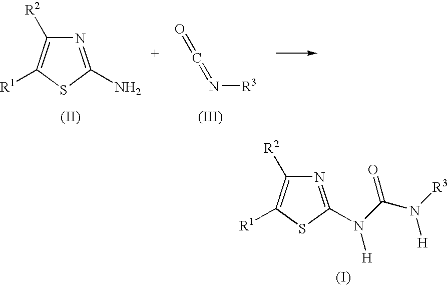
(ii) reacting a compound of formula II, wherein R² is hydrogen, with an activated carboxylic acid R³COL, wherein L is a leaving group such as Halo; 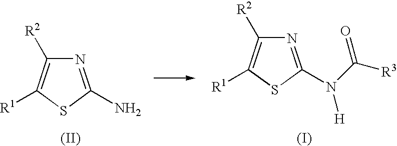
(iii) by using a carboxylic acid, R³COOH with an activating reagent such as N,N′-carbonyldiimidazole or N,N′-dicyclohexylcarbodiimide in a suitable solvent such as N,N-dimethylformamide or tetrahydrofuran and the reaction may be conducted at a temperature between +20° C. and +150° C.;
(iv) reacting a compound of formula IV, wherein R³ is C₁₋₆alkyl or C₀₋₆alkylaryl, with a compound of formula V, wherein R⁵ is C₁₋₆alkyl and Halo is chloro or bromo. [chemical structure]
